(19) United States
(12) Patent Application Publication
(10) Pub. No.: US 2021/0008358 A1
(43) Pub. Date: Jan. 14, 2021

(54) IMPLANT CAPABLE OF RELEASING DRUG, AND MANUFACTURING AND USAGE METHODS THEREFOR

(71) Applicant: KOLN HONG KONG LIMITED, Shatin, NT (HK)

(72) Inventors: Christian Xinshuo FANG, Shatin, NT (HK); Wing Fung Edmond YAU, Shatin, NT (HK); Sze Yi MAK, Shatin, NT (HK); Ching Hang Bob YUNG, Shatin, NT (HK)

(21) Appl. No.: 16/968,774

(22) PCT Filed: Jan. 29, 2019
(86) PCT No.: PCT/IB2019/050697

§ 371 Date: Aug. 10, 2020

(30) Foreign Application Priority Data
Feb. 12, 2018 (HK) 18102217.3

(51) Int. Cl. A61M 31/00; A61B 17/80; A61B 17/86

(52) U.S. Cl. CPC A61M 31/002; A61B 2017/00526; A61B 17/86; A61B 17/80

(57) ABSTRACT

The invention provides a drug-releasing implant, comprises the implant for fixing the surgical site, said implant is provided with the drug cavity for storing drug, said drug cavity is filled with liquid drug; said implant is further provided with micro pipeline, the micro pipeline passes through the drug cavity and the surface of the implant, the drug in the drug cavity is slowly released to the surgical site through the micro pipeline. The implant can effectively fix the bone at the surgical site (fracture site), after the surgery is completed, the drug set in implant's drug cavity slowly releases to the fracture site through micro pipeline, achieve local application of drug and reduce oral medication, so that decrease the fracture patients' medical expenses and psychological stresses, improve the utility of the device.

### TECHNICAL FIELD

The invention relates to the technical field of medical appliances, in particular to a drug-releasing implant, the manufacturing and using method of this implant.

### BACKGROUND OF THE INVENTION

At present, when the bone fracture occurs in a person, external fixation and internal fixation can be used as treatment. However, when the fracture is seriously damaged and the broken end is extremely unstable, the external fixation such as plastering and bracing may cause the fracture site to shift again. At this time, only the internal fixation can be used as treatment. The current method of internal fixation would depend on the condition of the patient. Intramedullary metal bone screw can be used to treat fractures by implanting the metal bone screw into the bone marrow, use the bone screw to fix the fractured bone; or use metal bone plate (orthopedic splint) and bone screw as fixing objects for the treatment of fractures, and use perforations on the metal bone plate to fix the fractured bone by the bone screw. In this way, it is necessary to drill the bone at the fracture site, then use one or more metal bone plates to connect the bones after the drilling is completed, and fix the metal bone plates with screws. When the fractured bone is healed, the metal bone screw or metal bone plate and screw should be decided whether to remove according to the situation. As the surgery should be taken, the fracture site is often very painful and might be inflamed especially 24 to 48 hours after the surgery. The pain is not only caused by the wound, but also comprising the psychological factor that affects the patient's rehabilitation progress. Generally, only internal drug can be used for pain relief and auxiliary treatment. Nevertheless, internal drug cannot avoid systemic administration and cannot meet the urgent needs of the majority of patients.

In addition, the capacity of implant's drug cavity is limited, it is required to inject the drug again after all the drugs are released. However, how to inject the drug to the drug cavity again conveniently and prevent the drug from leaking are not mentioned in the prior art.

### SUMMARY OF THE INVENTION

In view of the deficiency of above prior art, the invention provides a drug-releasing implant that is convenient for fixation and increases the therapeutic effectiveness, such as pain relief, anti-inflammatory, repair, cancer treatment, etc., and can perform local application of drug to the fracture site at the same time.

Another purpose of the invention is to provide the manufacturing method of above drug-releasing implant, so that customize appropriate implant according to the patient's fracture site, make the implant better fit with the fractured bone, in order to achieve good surgical effect.

One more purpose of the invention is to provide the using method of above drug-releasing implant.

The invention achieves above purposes in this way:

The drug-releasing implant, the implant is used to implant into a surgical site, said implant is provided with the drug cavity for storing drug, said drug cavity is filled with liquid drug; said implant is further provided with micro pipeline, the micro pipeline passes through the drug cavity and the surface of the implant, the drug in the drug cavity is slowly released to the surgical site through the micro pipeline.

Wherein, said micro pipeline comprises slow-release hole arranged on the wall, the slow-release hole is filled with porous spongy material for controlling the release speed of the drug, the drug in the drug cavity penetrates into the porous spongy material, then is released from the slow-release hole to the human body.

Wherein, drug injection structure is further provided, comprising the injection needle tubing, one end of which is the piercing end, the slow-release hole has the guiding tube for importing the piercing end, the piercing end pierces into porous spongy material through the guiding tube, and inject the drug into the drug cavity.

Wherein, said porous spongy material is filled and fixed in the drug cavity at the corresponding position of the slow-release hole. Wherein, said micro pipeline is the net structure set on the wall between the implant and the drug cavity.

Wherein, said implant is provided with screw mounting hole for fixing the implant to the surgical site, the locking screw can pass through the screw mounting hole to be fixedly connected to the surgical site.

The manufacturing method for the drug-releasing implant, wherein comprise the following steps:
A1. Obtain bone outline data of the surgical site;
A2. Calculate and generate the structural data of the implant according to the bone outline data, calculate the capacity of the drug cavity from the required drug dose after the surgery, and design the structural data of drug cavity and micro pipeline based on the structural data of the implant;
A3. Manufacture the implant by 3D printing method according to the structural data of implant, drug cavity and micro pipeline.

The using method for the drug-releasing implant, wherein comprise the following steps:
B1. Sterilize the implant and locking screw before the surgery;
B2. Anesthetize and sterilize the surgical site, then dissect the surgical site;
B3. Install the implant to the surgical site and adjust the implant to match the bone curvature of the surgical site, and then fix the implant to the surgical site with locking screw;
B4. Suture the surgical site;
Add the following Step C to B2 or B3: Inject the drug into the drug cavity.

Wherein, for said Step C, the wall between said implant and drug cavity is provided with injection hole, said drug is injected from the injection hole to the drug cavity by injector, then seal the injection hole.

Wherein, said Step C is to immerse the implant in the drug bath, the drug penetrates into the drug cavity through the micro pipeline.

Wherein, the drug cavity is set to a vacuum state and the implant is immersed in the drug bath, the drug penetrates into the drug cavity through the micro pipeline under the action of negative pressure.

Advantageous effect of the invention: The implant can effectively fix the bone at the surgical site (fracture site), after the surgery is completed, the drug set in implant's drug cavity slowly releases to the fracture site through micro pipeline, achieve local application of drug and reduce oral medication, so that decrease the fracture patients' medical expenses and psychological stresses, improve the utility of the device.

### BRIEF DESCRIPTION OF THE DRAWINGS

Below further describes the invention combining with the drawings:

FIG. 1 is the top view of the invention;

FIG. 2 is the left view of the invention;

FIG. 3 is the sectional view of the invention;

FIG. 4 is the drawing of one embodiment of micro pipeline;

FIG. 5 is the operation diagram of injecting drug into the implant in the embodiment of FIG. 4;

FIG. 6 is the operation steps of drug injection in the embodiment of FIG. 4;

FIG. 7 is the drawing of another embodiment of micro pipeline.

In the figures, 1. implant; 2. drug cavity; 3. micro pipeline; 4. screw mounting hole; 5. slow-release hole; 6. porous spongy material; 7. injection needle tubing; 8. guiding tube.

### DETAILED EMBODIMENT OF THE INVENTION

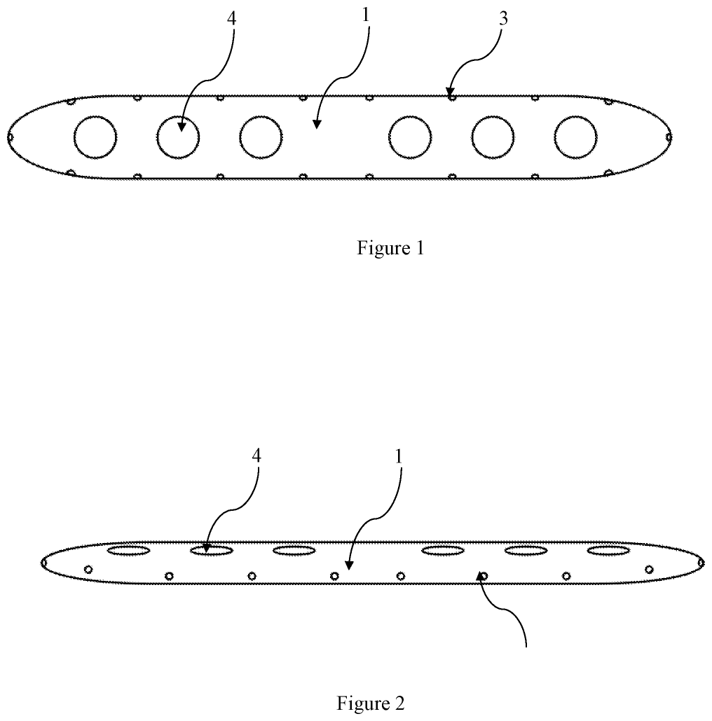

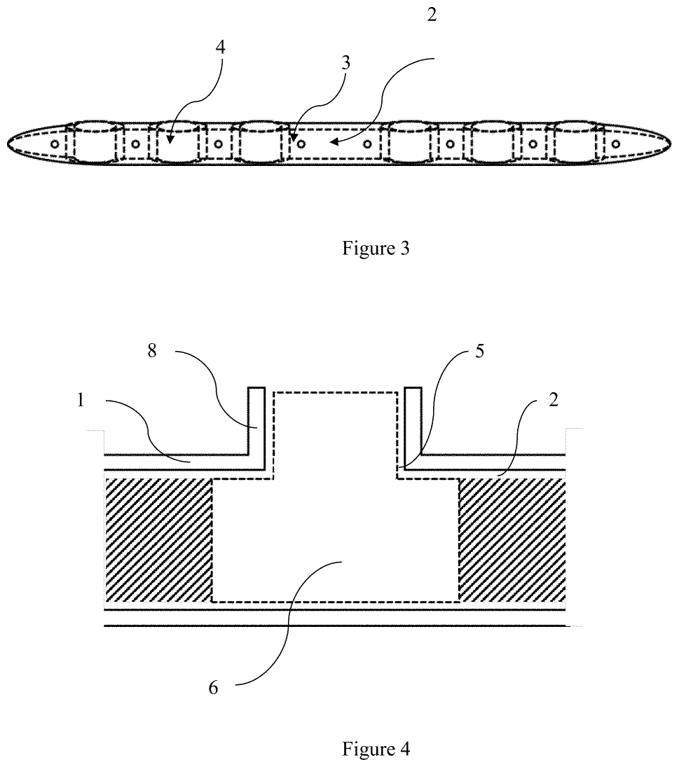

As shown in FIGS. 1-3, the drug-releasing implant comprises the implant (1) for fixing the surgical site, said implant (1) is provided with the drug cavity (2) for storing drug, said drug cavity (2) is filled with liquid drug; said implant (1) is further provided with micro pipeline (3), the micro pipeline (3) passes through the drug cavity (2) and the surface of the implant (1), the drug in the drug cavity (2) is slowly released to the surgical site through the micro pipeline (3). The implant (1) can effectively fix the bone at the surgical site (fracture site), after the surgery is completed, the drug set in implant's (1) drug cavity (2) slowly releases to the fracture site through micro pipeline (3), achieve local application of drug and reduce oral medication, so that decrease the fracture patients' medical expenses and psychological stresses, improve the utility of the device.

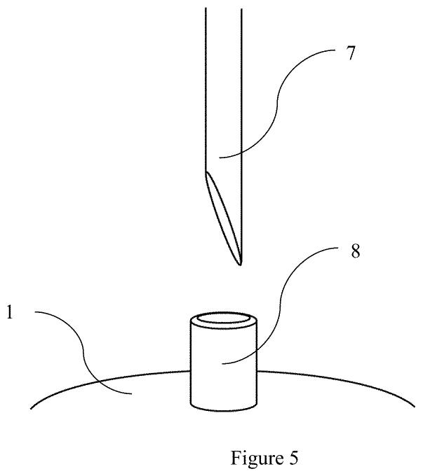

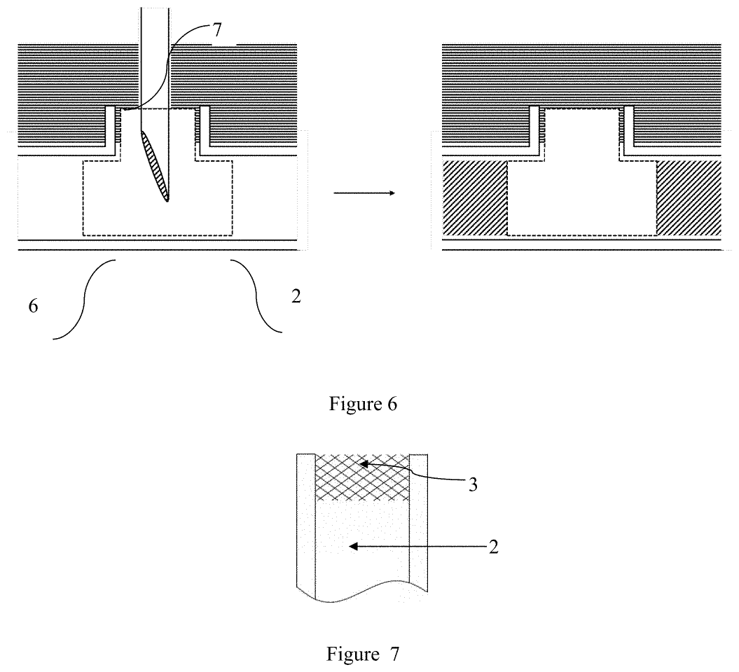

In order to release the drug in the drug cavity (2) conveniently, the micro pipeline (3) can be multiple structures, one is to provide slow-release hole (5) on the wall, the slow-release hole (5) is filled with porous spongy material (6) for controlling the release speed of the drug, the drug in the drug cavity (2) penetrates into the porous spongy material (6), then is released from the slow-release hole (5) to the human body, the user can select appropriate porous spongy material (6) to obtain different osmotic pressures according to the use condition of different drugs, so that control the release speed of the drug, please refer to FIG. 4. For above structure, drug injection structure is further provided as shown in FIG. 5 and FIG. 6, comprising the injection needle tubing (7), one end of which is the piercing end, the slow-release hole (5) has the guiding tube (8) for importing the piercing end, the piercing end pierces into porous spongy material (6) through the guiding tube (8), and inject the drug into the drug cavity (2). When the drug is required to inject to the implant (1) that has been implanted into the human body, the medical imaging equipment, such as X-ray, ultrasound imaging, magnetic resonance imaging, etc. should be used; during the operation, image the implant (1) and the injection needle tubing (7) so that the piercing end of the injection needle tubing (7) can be accurately pierced into the porous spongy material (6) of the implant (1) from the slow-release hole (5), and then the drug is injected into the porous spongy material through the injection needle tubing (7). Due to the drug injected pressure being relatively large, it can quickly pass through the porous spongy material and immerse into the drug cavity (2). After the drug injection operation is completed, the injection needle tubing (7) can be drawn out. The entire injection process is simple and quick.

In order to make the porous spongy material (6) be well fixed in the drug cavity (2), and prevent the drug from leaking directly to the slow-release hole (5), said porous spongy material (6) is filled and fixed in the drug cavity (2) at the corresponding position of the slow-release hole (5). The guiding tube (8) can guide the injection needle tubing (7) to accurately pierce into the slow-release hole (5), and the porous spongy material (6) extends into the guiding tube (8) at the same time; on the one hand, the porous spongy material (6) can be fixed by the guiding tube (8) to prevent it from moving and cause leakage of drug; on the other hand, when the drug is injecting, the porous spongy material (6) can effectively fix the piercing end.

Another one is the net structure set on the wall between the implant (1) and the drug cavity (2), please refer to Figure˜7.

In order to further control the release speed of the drug, the drug should be liquid or gel, and its viscosity is required to be 1×10⁻¹ to 1×10⁵ mPa·s.

In order to monitor the amount of drug used in the implant (1), the drug cavity (2) is provided with a sensor for monitoring the inventory of drug in the implant (1). The remaining amount of drug in the implant (1) can be learned in time. Thus, it can detect whether the release speed of the drug meets the predetermined target, and on the other hand, it can timely understand and notify the user to refill the drug in the implant (1).

Said implant (1) can be various devices used to fix the bone at the fracture site, comprising bone plate, bone screw (i.e. locking screws), etc. Wherein, the bone plate is used to connect and fix the fracture site, the bone screw is the device used to nail into the bone site to realize the connection between the bones or between the bone plate and the bone. The drug cavity (2) can be provided in both of the above, and the drug can be stored therein for slowly releasing to the fracture site after the surgery. In general, the overall size of bone plate is large, so the drug cavity (2) that can be provided is also large, and it is further provided with screw mounting hole at the same time, which is fixed to the bone by locking screw (i.e. bone screw). The size of the bone screw is small, only the small drug cavity (2) is provided, comprising bone marrow screw and bone screw.

The bone plate is taken as an example, detailed manufacturing steps and using method are described below.

As the outline of fractured bone in different sites and the site needing to be fixed are different, in order to make the bone plate better fit with the fracture site to achieve the most ideal surgery effect, the bone plate is manufactured by following steps: First of all, obtain the bone outline data of the surgical site through X-ray machine or magnetic resonance imaging equipment; then calculate and generate the structural data of the bone plate according to the bone outline data, and form the 3D model of bone plate through the computer, the wall thickness of bone plate can be 0.2-2 mm; the required drug dose in every surgery is different in actual situation, and the site to where the drug is released is also different, thus the capacity of drug cavity (2) can be calculated based on above situation, and design the structural data of drug cavity (2) and micro pipeline (3) based on the structural data of the bone plate, the capacity of drug cavity (2) is generally 0.1-10 ml, the volume ratio of drug cavity (2) to bone plate is 20%-80%, the final bone plate 3D model can be formed by the computer; finally, manufacture the bone plate by 3D printing method according to the structural data of the bone plate, drug cavity (2) and micro pipeline (3). Said bone plate or locking screw should use the medical-grade metal to be the manufacture material, the metals are ductile and flexible to match the curvature of the bones at different sites, comprising medical-grade stainless steel, cobalt alloy or titanium alloy. The bone plate has two main surfaces, one is toward the surface of the bone, another one is toward the surface of the muscle; wherein the surface facing the bone is smooth and has a curve surface that fits the bone, the central axis of the bone plate is provided with several sets of locking screw holes, locking screws can be used to fix the bone plate on the fractured bone.

The using method of drug-releasing bone plate, before the surgery, sterilize the bone plate and locking screw in order to prevent the bacteria or virus infection; then anesthetize and sterilize the surgical site, and dissect the surgical site; install the bone plate to the surgical site and adjust the bone plate to match the bone curvature of the surgical site, and then fix the bone plate to the surgical site with locking screw; the wall between bone plate and drug cavity (2) is provided with injection hole, said drug is injected from the injection hole to the drug cavity (2) by injector, then seal the injection hole; suture the surgical site. Under the action of the human body fluids, microtubules, penetration and diffusion, the drug in the drug cavity (2) is required to release to the surgical site within 24 to 48 hours after the surgery, thereby reducing the pain of fracture patients, and can perform local application of drug to the fracture site to reduce oral medication at the same time. Regarding the use of injection method mentioned in above using method, that injecting the drug into the drug cavity (2), can also use the penetration method, such as penetrating the bone plate into the drug bath, the drug penetrates into the drug cavity (2) through the micro pipeline (3). In addition, for expediting the penetration speed of drug, the drug cavity (2) can be set to a vacuum state and the bone plate is immersed in the drug bath, the drug penetrates into the drug cavity (2) through the micro pipeline (3) under the action of negative pressure.

Above contents are further described the invention combining with specific preferred embodiments, but cannot limit the embodiment of the invention to these only. For the common technicians who belong to the technical field of the invention, they can make some simple derivations or substitutions on the premise of not departing from the concept of the invention, it should be deemed as belonging to the protection scope of the invention.

1. The drug-releasing implant, wherein: the implant is used to implant into a surgical site, said implant is provided with the drug cavity for storing drug, said drug cavity is filled with liquid drug; said implant is further provided with micro pipeline, the micro pipeline passes through the drug cavity and the surface of the implant, the drug in the drug cavity is slowly released to the surgical site through the micro pipeline.

2. The drug-releasing implant of claim 1, wherein: Said micro pipeline comprises slow-release hole arranged on the wall, the slow-release hole is filled with porous spongy material for controlling the re ease speed of the drug, the drug in the drug cavity penetrates into the porous spongy material then is released from the slow-release hole to the human body.

3. (canceled)

4. The drug-releasing implant of claim 1, wherein: Said implant is provided with screw1 mounting hole for fixing the implant to the surgical site, the locking screw can pass through the screw mounting hole to be fixedly connected to the surgical site.

5. The manufacturing method for the drug-releasing implant of claim 1, wherein comprise the following steps:
A1. Obtain bone outline data of the surgical site;
A2. Calculate and generate the structural data of the implant according to the bone outline data, calculate the capacity of the drug cavity from the required drug dose after the surgery, and design the structural data of drug cavity and micro pipeline based on the structural data of the implant;
A3. Manufacture the implant by 3D printing method according to the structural data of implant, drug cavity and micro pipeline.

6. The using method for the drug-releasing implant of claim 3, wherein comprise the following steps:
B1. Sterilize the implant and locking screw before the surgery;
B2 Anesthetize and sterilize the surgical site, then dissect the surgical site;
B3. Install the implant to the surgical site and adjust the implant to match the bone curvature of the surgical site, and then fix the implant to the surgical site with locking screw;
B4. Suture the surgical site;
Add the following Step C to B2 or B3: Inject the drug into the drug cavity.

7. The using method for the drug-releasing implant of claim 7, wherein:
For said Step C, the wall between said implant 44 and drug cavity is provided with injection hole, said drug is injected from the injection hole to the drug cavity by injector, then seal the injection hole.

8. The using method for the drug-releasing implant of claim 7, wherein:
Said Step C is to immerse the implant W, in the drug bath, the drug penetrates into the drug cavity through the micro pipeline

9. The using method for the drug-releasing implant of claim 9, wherein:
The drug cavity is set to a vacuum state and the implant immersed in the drug bath, the drug penetrates into the drug cavity through the micro pipeline under the action of negative pressure.

10. The drug-releasing implant of claim 2, wherein: Drug injection structure is also provided, comprising the injection needle tubing, one end of which is the piercing end, the slow-release hole has the guiding tube for importing the piercing end, the piercing end pierces into porous spongy material through the guiding tube, and inject the drug into the drug cavity.

11. The drug injection structure of drug-releasing implant of claim 4, wherein: Said porous spongy material is filled and fixed in the drug cavity at the corresponding position of the slow-release hole.
